(19) United States
(12) Patent Application Publication
(10) Pub. No.: US 2016/0184001 A1
(43) Pub. Date: Jun. 30, 2016

(54) THERMAL ABLATION NEEDLE

(71) Applicant: METAL INDUSTRIES RESEARCH AND DEVELOPMENT CENTRE, Kaohsiung (TW)

(72) Inventors: Yi-San CHANG, Kaohsiung (TW); Tung-Chieh YANG, Kaohsiung (TW); Tsung-Chih YU, Kaohsiung (TW); Ho-Chung FU, Kaohsiung (TW)

(21) Appl. No.: 14/584,470

(22) Filed: Dec. 29, 2014

(51) Int. Cl. A61B 18/08; A61B 18/14

(52) U.S. Cl. CPC A61B 18/082; A61B 18/1477; A61B 2018/00577

(57) ABSTRACT

A thermal ablation needle of the present invention includes a magnetic section and a nonmagnetic section. The magnetic section includes a needle tip and a space that is spaced apart from the needle tip. The nonmagnetic section is connected to the magnetic section oppositely of the needle tip.

### FIELD OF THE INVENTION

The invention relates to a thermal ablation needle, more particularly to a thermal ablation needle detectable by an instrument during surgery.

### BACKGROUND OF THE INVENTION

Common types of treatments for cancer include surgical removal, minimally invasive ablation, chemotherapy and radiation therapy, depending on the location of the tumor, the stage of progression of the cancer, and whether the tumor cells have metastasized. The minimally invasive ablation treatment is usually used for smaller tumors in the earlier stages of cancer, with the less costly magnetic heating method being the most widely accepted approach for this type of treatment.

Referring to FIG. 1, a conventional thermal ablation apparatus includes a needle 11 and an electromagnetic coil device 12 having a coil 121 that is looped around the needle 11. The needle 11 is solid and has a magnetic section 111 that is formed with a needle tip 113, and a nonmagnetic section 112 that extends from the magnetic section 111 oppositely of the needle tip 113. In use, the electromagnetic coil device 12 controls a magnetic field to cause the magnetic section 111 to produce heat for ablation of a tumor. The incorporation of an ultrasound scanner into the process aids the user in positioning the tumor, yet the solid structure of the magnetic section 111 prevents detection by the scanner, making it impossible to pinpoint the exact location of the magnetic section 111 and to thereby ascertain that the needle tip 113 has been inserted accurately into the tumor.

### SUMMARY OF THE INVENTION

Therefore, the object of the present invention is to provide a thermal ablation needle that can alleviate at least one of the aforesaid drawbacks of the prior art.

Accordingly, a thermal ablation needle of the present invention comprises of a magnetic section and a nonmagnetic section. The magnetic section has a needle tip and a space that is spaced apart from the needle tip. The nonmagnetic section is connected to the magnetic section oppositely of the needle tip.

### BRIEF DESCRIPTION OF THE DRAWINGS

Other features and advantages of the present invention will become apparent in the following detailed description of the embodiments with reference to the accompanying drawings, of which:

FIG. 1 is a perspective view of a conventional thermal ablation needle in use;

FIG. 2 is a fragmentary sectional view of a first embodiment of a thermal ablation needle according to the invention;

FIG. 3 is a perspective view of the first embodiment;

FIG. 4 is a fragmentary sectional view of a second embodiment of the thermal ablation needle according to the invention;

FIG. 5 is a fragmentary sectional view of a third embodiment of the thermal ablation needle according to the invention; and

FIG. 6 is a fragmentary sectional view of a fourth embodiment of the thermal ablation needle according to the invention.

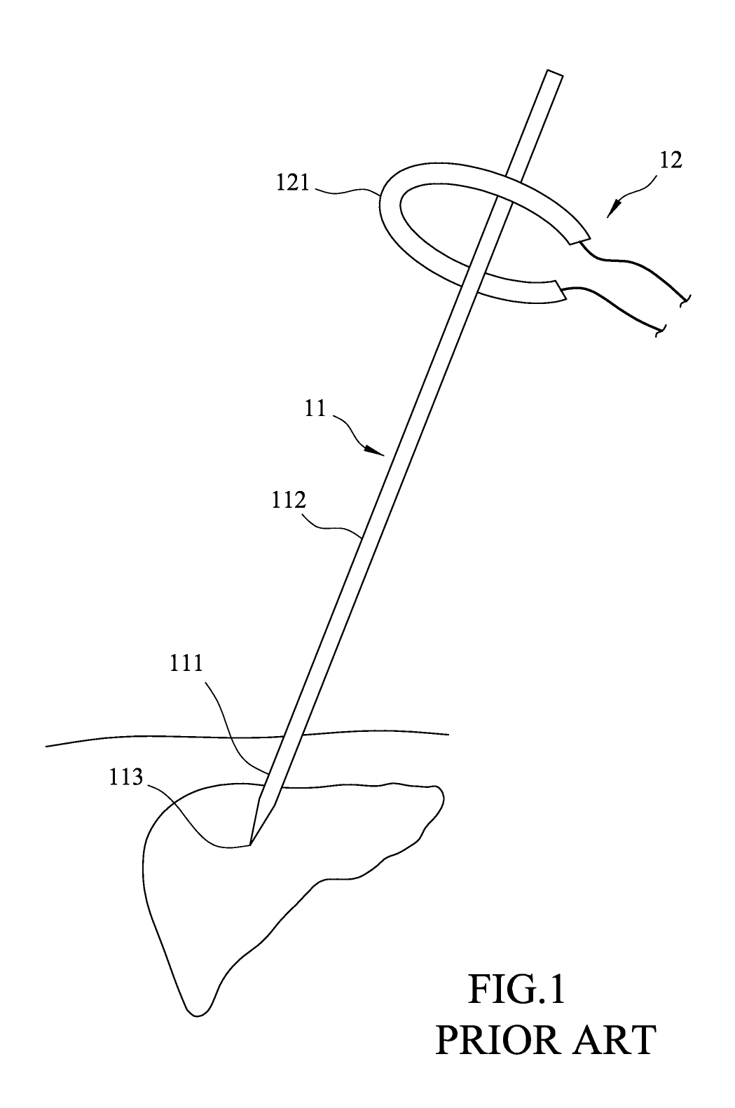

### DETAILED DESCRIPTION OF THE EMBODIMENTS

Before the present invention is described in greater detail, it should be noted that like elements are denoted by the same reference numerals throughout the disclosure.

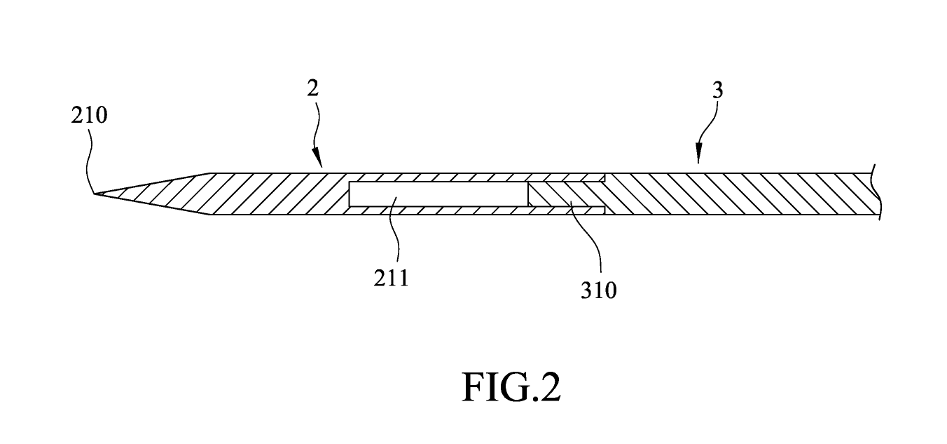

Referring to FIG. 2, a first embodiment of a thermal ablation needle according to the present invention is detectable by an instrument during surgery, and includes a magnetic section 2 and a nonmagnetic section 3. The magnetic section 2 has a needle tip 210 and a first space 211 that is spaced apart from the needle tip 210. The nonmagnetic section 3 is connected to the magnetic section 2 oppositely of the needle tip 210. In this embodiment, the nonmagnetic section 3 has a connection part 310 that connects the nonmagnetic section 3 to the magnetic section 2. The magnetic section 2 is formed as one piece, and is sleeved around the connection part 310 of the nonmagnetic section 3. It should be noted, however, that the structural configuration of the thermal ablation needle is not limited to the disclosure herein.

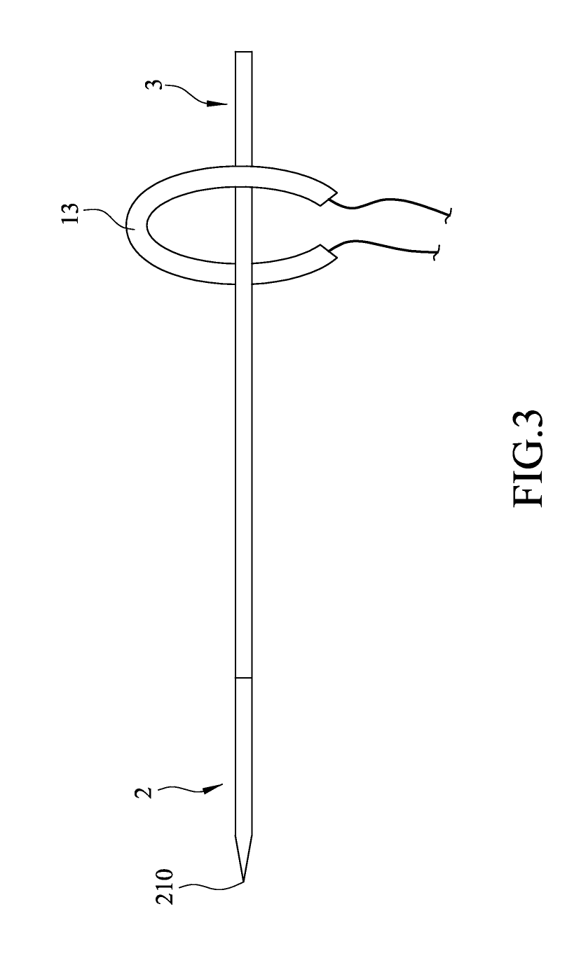

Referring to FIG. 3, when operating with the thermal ablation needle, the needle tip 210 is inserted into a tumor (not shown), and an electromagnetic coil 13 is looped around the thermal ablation needle to convert electric current from an exterior source into a high frequency alternating magnetic field that is introduced into the magnetic section 2 to induce eddy currents. A power directly proportionate to magnitude of the eddy currents is generated from the electric resistance present in the magnetic section 2 and dissipates in the form of heat. When the heat from the magnetic section 2 inserted in the tumor reaches an ablating temperature, the tumor cells will then be killed. Since the nonmagnetic section 3 is made of magnetically impermeable material and may serve as insulation, the healthy tissues around the tumor are thereby protected from the ablating heat.

In order to more accurately pinpoint the location of the tumor, an ultrasound scanner (not shown) is incorporated into the thermal ablation process. The first space 211 of the magnetic section 2 allows a white spot to be shown on a sonogram produced by the ultrasound scanner so that the exact location of the magnetic section 2 can be identified for accurate insertion of the magnetic section 2 into the tumor.

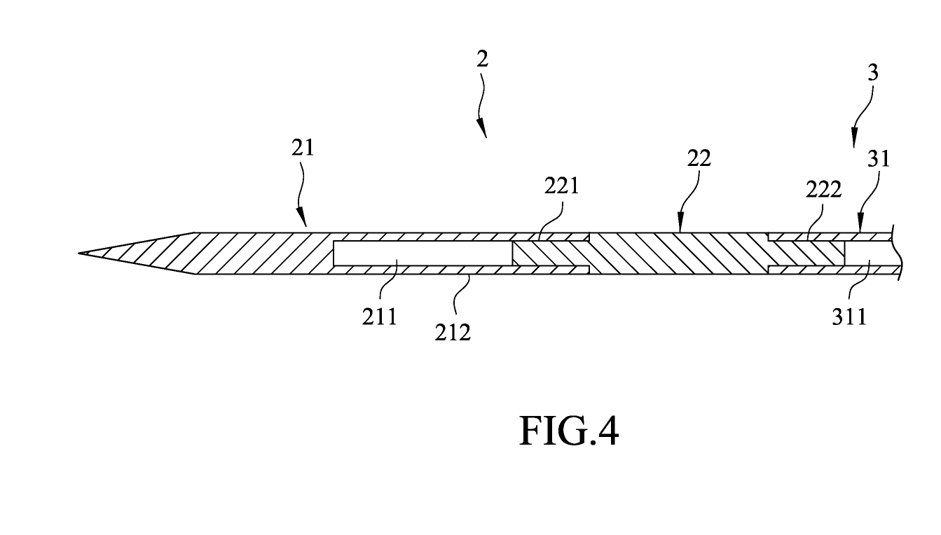

Referring to FIG. 4, a second embodiment of a thermal ablation needle according to the present invention has a structure similar to that of the first embodiment. The difference between the second embodiment and the first embodiment resides in the configuration of the magnetic section 2 and the nonmagnetic section 3. In the second embodiment, the magnetic section 2 includes a first portion 21 having a first tubular wall 212 that defines the first space 211, and a second portion 22 having a first insert part 221 that is inserted into the first tubular wall 212 and that closes off the first space 211. The nonmagnetic section 3 has a second tubular wall 31 defining a second space 311 that is spaced apart from the first space 211. The second portion 22 of the magnetic section 2 further has a second insert part 222 that is opposite to the first insert part 221 and that is sleeved into the second tubular wall 31.

When incorporating the ultrasound scanner to locate the tumor, the second space 311 of the nonmagnetic section 3 cooperates with the first space 211 of the magnetic section 2 to form a straight, white line on the sonogram that allows the user to clearly discern between the thermal ablation needle and any misleading random white spots that may appear due to interference.

This two-piece design of the magnetic section 2 provides an alternative configuration for the thermal ablation needle.

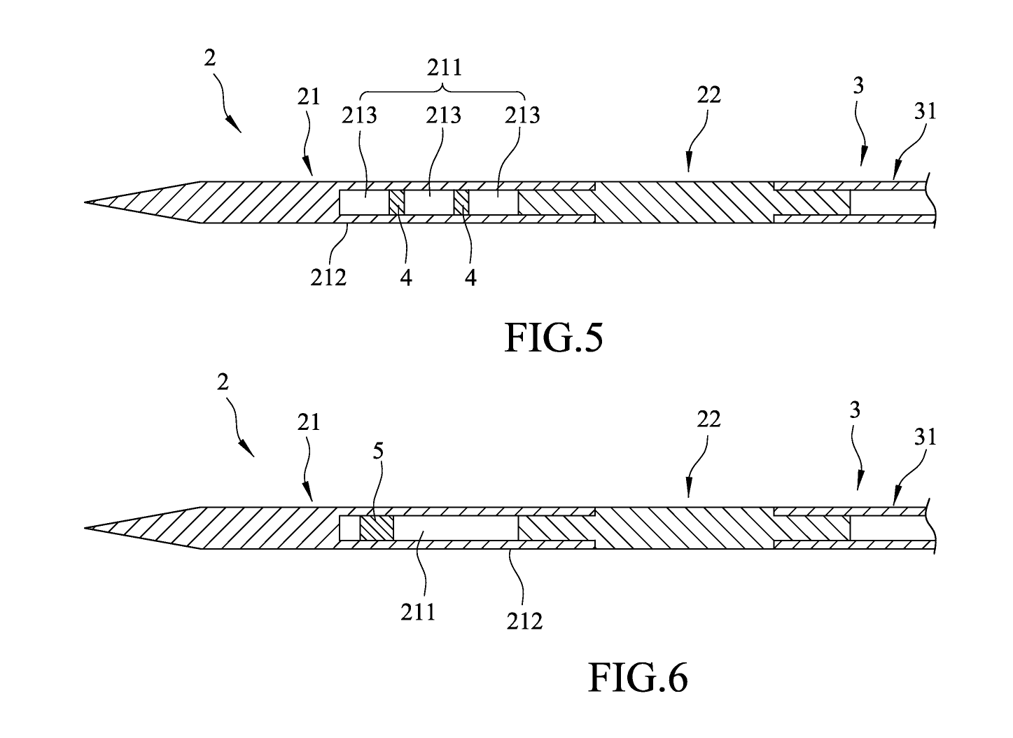

Referring to FIG. 5, a third embodiment of a thermal ablation needle according to the present invention has a structure similar to that of the first embodiment, except that the thermal ablation needle further includes a plurality of separation members 4 disposed in the first space 211 for dividing the first space 211 into a plurality of hollow regions 213. When incorporating the ultrasound scanner, the separation members 4 serve to have the thermal ablation needle display on the sonogram as a banded, white line that allows the user to clearly discern between the thermal ablation needle and any random white spots.

Referring to FIG. 6, a fourth embodiment of a thermal ablation needle according to the present invention further includes a magnetic element 5 disposed in but not completely filling the first space 211. The magnetic element 5 has a permeability coefficient larger than that of the magnetic section 2. The effect of the foregoing is that the magnetic field may be converged to the vicinity of the magnetic section 2, and the eddy currents through electromagnetic induction can be generated more efficiently, thereby improving the heating efficiency of the magnetic section 2.

In sum, the first space 211 of the thermal ablation needle allows the user to easily identify the exact location of the magnetic section 2 with the ultrasound scanner, and may be disposed with the magnetic element 5 to increase the heating efficiency of the magnetic section 2.

While the present invention has been described in connection with what are considered the most practical embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A thermal ablation needle detectable by an instrument during surgery, comprising:
a magnetic section having a needle tip, and a first space that is spaced apart from said needle tip; and
a nonmagnetic section connected to said magnetic section oppositely of said needle tip.

2. The thermal ablation needle as claimed in claim 1, wherein said nonmagnetic section has a connection part, said connection part and said magnetic section being sleeved one around the other.

3. The thermal ablation needle as claimed in claim 1, wherein said magnetic section includes:
a first portion having a first tubular wall that defines said first space; and
a second portion having a first insert part that is inserted into said first tubular wall and that closes said first space.

4. The thermal ablation needle as claimed in claim 3, wherein said nonmagnetic section has a second space that is spaced apart from said first space.

5. The thermal ablation needle as claimed in claim 4, wherein:
said second portion of said magnetic section further has a second insert part that is opposite to said first insert part; and
said nonmagnetic section further has a second tubular wall that defines said second space and that is sleeved around said second insert part of said second portion of said magnetic section.

6. The thermal ablation needle as claimed in claim 1, further comprising a magnetic element disposed in said first space without completely filling said first space, said magnetic element having a permeability coefficient larger than that of said magnetic section.

7. The thermal ablation needle as claimed in claim 1, further comprising at least one separation member disposed in said first space to divide said first space into a plurality of hollow regions.
